(19) United States
(12) Patent Application Publication
(10) Pub. No.: US 2024/0217904 A1
(43) Pub. Date: Jul. 4, 2024

(54) METHOD FOR PREPARING CEDRENE DIOL

(71) Applicant: SYMRISE AG, Holzminden (DE)

(72) Inventors: Arne WEBER, Hannover (DE); Thomas WERNER, Rostock (DE); Aiga GRANDANE, Valdlauci (LV); Vivian STEFANOW, Rostock (DE); Oliver LENZ, Uslar (DE)

(21) Appl. No.: 18/556,418

(22) PCT Filed: Apr. 22, 2021
(86) PCT No.: PCT/EP2021/060525

§ 371 Date: Oct. 20, 2023

(51) Int. Cl. C07C 29/48; C07C 29/84

(52) U.S. Cl. CPC C07C 29/48; C07C 29/84

(57) ABSTRACT

The present invention relates to a method of preparing cis-cedrene diol comprising providing alpha-cedrene in a solvent comprising or consisting of water and at least one further protic solvent, adding potassium permanganate and a base and, optionally, removing the further protic solvent from the reaction mixture.

The present invention relates to a method for preparing cis-cedrene diol comprising providing alpha-cedrene in a solvent comprising or consisting of water and at least one further protic solvent, adding potassium permanganate and a base and, optionally, removing the further protic solvent from the reaction mixture.

Cedrene diols are important starting materials for the production of high-quality fragrances. Due to its outstanding odor properties as an acetonide, cis-cedrene diol is of particular interest. The production of cis-cedrene diol is usually carried out from cedrene in a two-step process. First, cedrene is oxidized to cedrene epoxide and this epoxide is then opened to the diol. When the epoxide is opened, however, by-products are also formed (cedralone, trans-cedrene diol).

Semmler and Hoffmann report the oxidation of cedrene with potassium permanganate to cedrene diol (F. W. Semmler, A. Hoffmann, Chem. Ber., 1907, 40, 3521). Cedrene is dissolved in aqueous acetone and finely powdered potassium permanganate is gradually introduced. Purification is by extraction with ether followed by distillation of the ether. However, the yield based on the cedrene used is only 12-15% and the method described is therefore not suitable for economical synthesis.

It was an object of the present invention to provide a method for the direct preparation of cis-cedrene diol from cedrene without an intermediate step, in which yields are achieved which permit economic application.

The object is solved by a method for preparing cis-cedrene diol comprising the steps:
i) providing alpha-cedrene in a solvent comprising or consisting of water and at least one further protic solvent, preferably selected from the group consisting of secondary and tertiary alcohols, more preferably selected from the group consisting of tert-butanol and isopropanol,
ii) adding potassium permanganate and a base to the solution from step i), and, optionally
iii) removing the further protic solvent from the reaction mixture of step ii).

It was surprisingly found that the use of a mixture comprising or consisting of water and at least one further protic solvent and the addition of potassium permanganate together with a base could significantly increase the yield of cis-cedrene diol. Thus, yields of over 80% and even 90% could be achieved up to a 100 g scale. This could also be reproduced on a 150 kg scale. The reaction according to the invention is shown in Scheme 1.

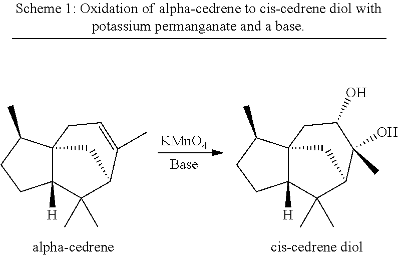

The presence of the base prevents the undesired formation of cedralone. Cedrene diol is significantly more unstable under acidic conditions than under basic ones (see Scheme 2).

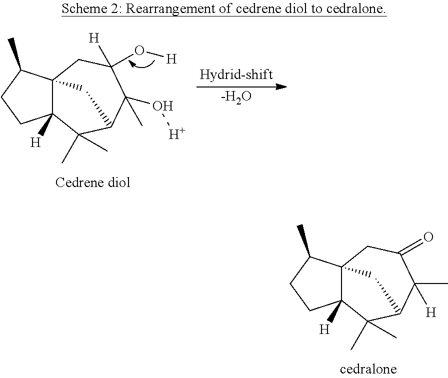

Moreover, the base favors the formation of the diol because it accelerates the cleavage of the manganese from the manganese-cedrene diol complex, as shown in Scheme 3, and thus further oxidation of the diol cannot occur (Wiberg and Saegebarth, J. Am. Chem. Soc., 1957, 79, 11, 2822-2824).

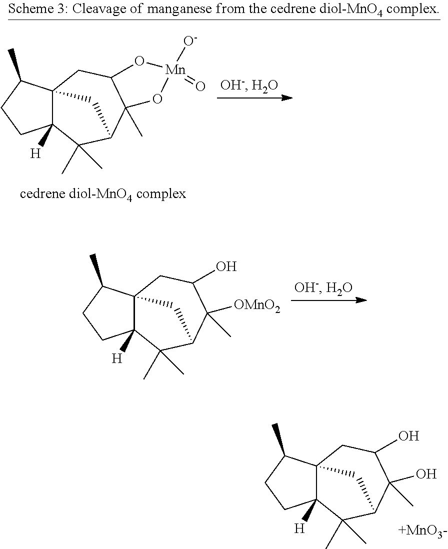

In the context of the present invention, a protic solvent means any solvent which has a functional group from which hydrogen can be split off as a proton. Since water can dissociate into H⁺ and OH⁻, it also constitutes a protic solvent. Further protic solvents include primary, secondary and tertiary alcohols and carboxylic acids. Alkanes, ethers, esters, ketones and aldehydes, on the other hand, do not represent protic solvents in the sense of the present invention.

In the context of the present invention, a base means a compound that forms hydroxide ions in aqueous solution. Suitable bases for use in step ii) are described below.

Secondary and tertiary alcohols and especially tert-butanol and isopropanol have proved to be advantageous. Secondary and tertiary alcohols dissolve cedrene well and no phase separation occurs when an aqueous solution of potassium permanganate is added. These solvents are also largely inert under the reaction conditions, so they are not oxidized by the potassium permanganate, thus avoiding the need for a larger amount of oxidizing agent. This is particularly true for tertiary alcohols such as tert-butanol, but only to a limited extent for isopropanol, since oxidation to acetone is possible. However, isopropanol is still completely miscible with water, so that good yields are still obtained. The solvents mentioned can also be easily removed by distillation, as they are more volatile than water.

In a preferred embodiment, the method described above additionally comprises the steps:
iv) extracting the resulting cis-cedrene diol from the reaction mixture of step iii), and
v) Removing the extracting agent from step iv) and obtaining cis-cedrenediol.

The resulting cis-cedrene diol can be obtained by extraction. In this context, it is important that in step iii) the further protic solvent is removed as completely as possible so that it does not interfere with the phase separation during the subsequent extraction. Suitable extraction solvents are described below.

Further preferred is a process as described above, wherein in step i) the alpha-cedrene has a purity of at least 70%, preferably at least 80%, more preferably at least 90% and/or wherein the alpha-cedrene is distilled prior to step i).

In step i), for example, cedar oil can be used in which alpha-cedrene is present at about 70%. If the alpha-cedrene, e.g. in the form of cedar oil, is distilled before step i), a purity of more than 90% can be achieved. If less pure alpha-cedrene is used, more potassium permanganate and base are required and dosing must be carried out at a lower temperature.

In another preferred embodiment of the method described above, in step i) the alpha-cedrene is provided in a weight ratio of 1:5 to 1:10 alpha-cedrene to the further protic solvent, preferably in a weight ratio of 1:6 to 1:9 alpha-cedrene to the further protic solvent, particularly preferably in a weight ratio of 1:7 to 1:8 alpha-cedrene to the further protic solvent and/or in step i) the alpha-cedrene is provided in a weight ratio of 1:1 to 1:6 alpha-cedrene to water, preferably in a weight ratio of 1:2 to 1:5 alpha-cedrene to water, particularly preferably in a weight ratio of 1:3 to 1:4 alpha-cedrene to water.

It has been shown that with less further protic solvent in relation to alpha-cedrene, the conversion is incomplete because cedrene oils out and separates. If less water is used in relation to cedrene, complete conversion occurs but with less yield. If the specified weight ratios are maintained, the conversion is complete and the yield is good.

Also preferred is a method as described above in which the addition in step ii) takes place over a period of 30 minutes to 10 hours, preferably 3 to 7 hours, more preferably 4 to 6 hours.

In the method according to the invention, a good conversion and good yields can be achieved after only a few hours. However, if dosing is faster, the GC purity (purity according to gas chromatography) of the final product decreases. Good results are obtained from 3 to 4 hours dosing time. However, dosing in the method according to the invention can also be carried out over a period longer than 10 hours, e.g. over 15 or 20 hours.

In another preferred embodiment of the method described above, the base is an inorganic base, preferably selected from the group of alkali and alkaline earth hydroxides and alkali and alkaline earth carbonates, particularly preferably selected from NaOH, KOH, K₂CO₃ and CaCO₃.

Further preferred is also a method as described above, wherein in step ii) the potassium permanganate is added in an amount of 0.8 to 5 equivalents relative to the alpha-cedrene provided in step i), preferably in an amount of 1 to 3 equivalents, particularly preferably in an amount of 1.2 to 1.5 equivalents and/or wherein the base is added in an amount of 0.5 to 3 equivalents relative to the alpha-cedrene provided in step i), preferably in an amount of 1 to 1.5 equivalents, particularly preferably in an amount of 1.2 to 1.3 equivalents.

By maintaining the molar ratios of the reagents specified above, i.e. potassium permanganate and base to alpha-cedrene used, particularly good yields were obtained.

In another preferred embodiment of the method described above, in step ii) a mixture comprising or consisting of potassium permanganate, base and water is added to the solution from step i), preferably the mixture is continuously stirred during addition and/or a gas is passed through the mixture.

The addition of potassium permanganate in aqueous solution rather than as a solid has the advantage that the concentration of potassium permanganate is not increased so much locally. In addition, the locally increased concentration also decomposes more quickly when potassium permanganate that is already dissolved is added than when potassium permanganate is added as a solid. A locally increased concentration can promote side reactions that result in more highly oxidized products.

Preferably, in the mixture added to the solution of step i) in step ii), the water is present in a weight ratio of 1:5 to 1:20 potassium permanganate to water, preferably 1:10 to 1:15 potassium permanganate to water, more preferably 1:12 to 1:14 potassium permanganate to water.

Since the mixture is at the solubility limit of the potassium permanganate, it is advantageous to stir the mixture constantly during addition, or to pass a gas, e.g. nitrogen, through the mixture. The stirring or the gas bubbles keep the mixture in motion and the potassium permanganate remains in solution.

In one embodiment of the method described above, in step i) the alpha-cedrene is provided in the solvent at a temperature in the range of 5 to 40° C., preferably 15 to 30° C., more preferably 18 to 22° C. and/or in step ii) the potassium permanganate and the base are added at a temperature of 10 to 40° C., preferably 15 to 30° C., more preferably 20 to 24° C.

The method according to the invention can be carried out in a relatively wide temperature range, in particular also at temperatures around room temperature, so that no heating or cooling is necessary. If dosing is performed at higher temperatures, the GC purity of the final product decreases. Dosing at lower temperature (about 10° C.) also works, but does not improve the result of the reaction.

According to another preferred embodiment of the method described above, the removal of the further protic solvent in step iii) is carried out by distillation.

As already mentioned above, for the purification of the product by extraction, it is important that the further protic solvent in step iii) is removed as completely as possible so that the phase separation is not disturbed. Removal by distillation achieves particularly good results. If tert-butanol is used as a further protic solvent, it is preferably distilled off as an azeotrope containing 10 to 30% water, particularly preferably as an azeotrope containing 15 to 25% water.

After distillation, it is advantageous to cool the distillation residue to about 40° C. before adding the extractant. This ensures that the cedrene diol is not partially destroyed again (formation of cedralone) when the mixture is acidified in the following step. The dosage of the sulfuric acid and especially the sodium hydrogen sulfite solution are also slightly exothermic.

In a further embodiment of the method according to the invention, the extractant used in step iv) is an aprotic extractant, preferably selected from the group consisting of ethers and esters, particularly preferably selected from the group consisting of ethyl acetate and methyl tert-butyl ether.

In the context of the present invention, an aprotic solvent means a solvent which does not have a functional group from which hydrogen can be split off as a proton. Aprotic solvents are, for example, aliphatic or aromatic alkanes or alkenes, esters, ethers, ketones or aldehydes. In particular, the solubility of cedrene diol is very good in ethyl acetate, so that a high space-time yield is obtained.

Preferably, in the method described above in step iv), the extractant is used in a weight ratio of 1:2 to 1:20 of the alpha-cedrene provided in step i) to the extractant, preferably in a weight ratio of 1:5 to 1:12 of the alpha-cedrene provided in step i) to extractant, particularly preferably in a weight ratio of 1:8 to 1:9.5 of the alpha-cedrene provided in step i) to extractant.

To extract the product completely, certain amounts of extractant are required. The extractant can also be added in two or more portions and the extractant phase separated in between to increase the space-time yield of the process.

As a further measure to improve extraction efficiency, sulfuric acid can be added. However, if the temperature is too warm (>40° C.), slightly more cedralone is formed as a by-product. In addition, a sodium hydrogen sulfite solution can also be added, which acts as a reducing agent and reduces all the manganese contained in the mixture to manganese(II), which is readily soluble in water. Protons are consumed in this redox reaction, so it runs preferentially in an acidic environment. The sulfite is oxidized to sulfate in the process. Towards the end of dosing, the temperature can be raised to approx. 50 to 55° C. by means of a trace heating system. This produces two clear phases and the increased temperature results in better phase separation. Some cedrene diol may already precipitate at this stage, which can be brought back into solution by further addition of extractant or an increase in temperature. The aqueous phase can then be separated and the extraction repeated with another portion of extractant. By adding soda ash and water, last residues of sulfuric acid can be removed or neutralized from the organic phase. To increase the storage stability of the cedrene diol, no traces of acid should remain in the cedrene diol. In addition, washing with sodium chloride/water can still be performed to ensure that no acid residues remain in the cedrene diol and that the resulting cedrene diol solution is approximately neutral. The sodium chloride allows the phases to separate better/faster and also less organic compound enters the wastewater. The product may already partially precipitate in the extractant phase during cooling. By removing the extractant, the cis-cedrene diol is obtained in a GC purity of over 90% and in yields of over 70%, preferably over 80%, particularly preferably over 90%.

The present invention also relates to the use of a mixture comprising or consisting of water and at least one other protic solvent, preferably selected from the group consisting of secondary and tertiary alcohols, more preferably selected from the group consisting of tert-butanol and isopropanol, as a solvent in the oxidation of alpha-cedrene with potassium permanganate, preferably in a method as described above.

By using the mixture comprising or consisting of water and at least one further protic solvent, the yield of cis-cedrene diol could be decisively increased. This makes it possible for the first time to obtain cis-cedrene diol in one step by oxidation with potassium permanganate in a yield that permits economic application.

Depending on the starting material, two isomers of cis-cedrene diol can be formed in the method according to the invention (compounds of the formulae (I) and (II), see below). In this case, the two isomers are each present in a characteristic amount, with compound (I) clearly predominating. By way of example, a mixture is formed in which the compound of formula (I) is present in an amount of 70 to 99% by weight, particularly preferably 85 to 95% by weight, and/or the compound of formula (II) is present in an amount of 0.1 to 2% by weight, particularly preferably 0.3 to 1.5% by weight, in each case based on the total weight of the mixture.

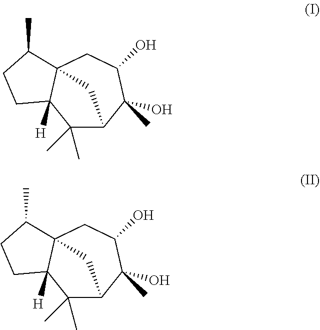

In the following, the invention is further characterized by means of examples, whereby these examples are not to be understood as limiting.

### Example 1: Preparation of cis-cedrene diol

1) alpha-cedrene (1.00 g, 1.07 mL, 4.89 mmol) is dissolved in a mixture of tBuOH (10 mL) and H₂O (5 mL).

2) The colorless solution is cooled to 0° C. and a mixture of KMnO₄ (1.12 g, 7.09 mmol) and NaOH (0.254 g, 6.36 mmol) in H₂O (15 mL) is added slowly (over a period of 45 min) at 0° C. The reaction temperature is kept below 10° C. After addition, the reaction mixture is stirred at 0° C. for 25 min.

3) After complete conversion of the alpha-cedrene (the reaction is monitored by DC (thin layer chromatography)), the reaction mixture is filtered through a celite filter (h=1.5 cm, d=5 cm). The celite filter is washed with EtOAc (30 mL).

4) The filtrate is saturated with NaCl and extracted with EtOAc (3×20 mL). The organic phase is washed with 10% Na₂S₂O₃ solution (1×20 mL), dried over Na₂SO₄ and the volatile components are evaporated to give cis-cedrenediol as a white solid (893 mg, 3.75 mmol, 77%). Rf (SiO₂, CH:EtOAc, 3:1)=0.18.

### Example 2: Preparation of cis-cedrene diol

1) Cedrene (purity about 93%, 100 g, 0.46 mmol, 1.0 eq) is placed in tert-butanol (720 g) and water (360 g) and tempered to 20° C.

2) Within about 5 h, a mixture of potassium permanganate (98 g, 0.62 mol, 1.35 eq), water (1320 g), and NaOH (22.4 g, 0.56 mol, 1.22 eq) is uniformly dispensed at an internal temperature of 20-24° C. (slightly exothermic reaction). Nitrogen is continuously passed through the mixture and/or stirring is continuous. A brown mixture with precipitate is formed.

3) The tert-butanol is distilled off as an azeotrope with approx. 20% water at 140 mbar. The trace heating is set to 80° C. A total of approx. 910 g of distillate is obtained. The tert-butanol obtained can be used again for the reaction.

4) Then cooled to 40° C. and ethyl acetate (700 g) added.

5) Sulfuric acid (40%, 200 g) is added at 40° C.

6) At about 40° C. sodium hydrogen sulfite solution (40%, 215 g) is added and towards the end of the dosing the heat tracing is already raised to 55° C. Two clear phases are formed and some cedrene diol precipitates.

7) The aqueous phase (approx. 2300 g) is separated.

8) Ethyl acetate (700 g), soda ash (40 g) and water (360 g) are added. This dissolves the precipitated cedrene diol again.

9) It is tempered to approx. 55° C. and mixed well.

10) The aqueous phase is separated.

11) Sodium chloride (30 g) and water (270 g) are added.

12) It is tempered again to 55° C. and mixed well.

13) The aqueous phase is separated.

The product subsequently precipitates in ethyl acetate; the cedrenediol partially precipitates on cooling. Removing the solvent yields about 102 g of cedrene diol of formula (I) (purity according to GC about 93%, about 0.40 mmol, about 87% yield). Other substances contained according to GC are: Cedrene (<1%), Cedralone (<1%), Cedrenol (about 1%) and the isomer of formula (II) (<1%).

1. A method for preparing cis-cedrene diol comprising:
(i) providing a solution of alpha-cedrene in a solvent, wherein the solvent comprises water and at least one further protic solvent;
(ii) adding potassium permanganate and a base to the solution and forming a reaction mixture comprising cis-cedrene diol;
(iii) optionally, removing the further protic solvent from the reaction mixture.

2. The method of claim 1, wherein the method further comprises:
iv) extracting the cis-cedrene diol from the reaction mixture with an extractant; and
v) removing the extractant and obtaining cis-cedrene diol.

3. The method of claim 1, wherein the alpha-cedrene has a purity of at least 70%.

4. The method of claim 1, wherein the alpha-cedrene and the further protic solvent are in a weight ratio of 1:5 to 1:10 (alpha-cedrene:further protic solvent) and the alpha-cedrene and the water are in a weight ratio of 1:1 to 1:6 (alpha-cedrene:water).

5. The method of claim 1, wherein the potassium permanganate and the base are added to the solution over a period of 30 minutes to 10 hours.

6. The method of claim 1, wherein the base is an inorganic base.

7. The method of claim 1, wherein the potassium permanganate is added to the solution in an amount of 0.8 to 5 equivalents, relative to the alpha-cedrene provided in (i) and/or the base is added to the solution in an amount of 0.5 to 3 equivalents relative to the alpha-cedrene provided in (i).

8. The method of claim 1, wherein a mixture comprising the potassium permanganate, base, and water is added to the solution.

9. The method of claim 8, wherein the water and the potassium permanganate in the mixture are in a weight ratio of 1:5 to 1:20 (potassium permanganate:water).

10. The method of claim 1, wherein the solution of the alpha-cedrene in the solvent is provided at a temperature of 5 to 40° C.

11. The method of claim 1 comprising: (iii) removing the at least one further protic solvent from the reaction mixture, wherein the removal is carried out by distillation.

12. The method of claim 2, wherein the extractant is an aprotic extractant.

13. The method of claim 2, wherein the extractant is used in a weight ratio of 1:2 to 1:20 of the alpha cedrene provided in (i) (extractant:alpha-cedrene).

14. The method of claim 1, wherein the at least one further protic solvent is selected from secondary alcohols, tertiary alcohols, or mixtures thereof.

15. The method of claim 1, wherein the alpha-cedrene and the at least one further protic solvent are in a weight ratio of 1:6 to 1:9 (alpha-cedrene:at least one further protic solvent) and the alpha-cedrene and the water are in a weight ratio of 1:2 to 1:5 (alpha-cedrene:water).

16. The method of claim 1, wherein the base is selected from alkali earth hydroxides, alkaline earth hydroxides, alkali earth carbonates, alkaline earth carbonates, or mixtures thereof.

17. The method of claim 1, wherein the potassium permanganate is added to the solution in an amount of 0.8 to 5 equivalents, relative to the alpha-cedrene provided in (i), and/or the base is added to the solution in an amount of 1 to 1.5 equivalents relative to the alpha-cedrene provided in (i).

18. The method of claim 8, wherein the water and the potassium permanganate in the mixture are in a weight ratio of 1:10 to 1:15 (potassium permanganate:water).

19. The method of claim 1, wherein the solution of the alpha-cedrene in the solvent is provided at a temperature of 15 to 30° C.

20. The method of claim 2, wherein the extractant is selected from ethyl acetate, methyl tert-butyl ether, and mixtures thereof.
